(19) United States
(12) Patent Application Publication
(10) Pub. No.: US 2019/0381299 A1
(43) Pub. Date: Dec. 19, 2019

(54) METHOD FOR MAKING MICRONEEDLES

(71) Applicant: SABIC GLOBAL TECHNOLOGIES B.V., Bergen op Zoom (NL)

(72) Inventors: Johannes DE BROUWER, Oisterwijk (NL); Herwig JUSTER, Bergen Op Zoom (NL)

(21) Appl. No.: 16/481,526

(22) PCT Filed: Jan. 31, 2018
(86) PCT No.: PCT/IB2018/050603

§ 371 Date: Jul. 29, 2019

Related U.S. Application Data
(60) Provisional application No. 62/452,557, filed on Jan. 31, 2017.

(51) Int. Cl. A61M 37/00; B29C 43/46; B29C 43/52

(52) U.S. Cl. CPC A61M 37/0015; B29C 43/46; B29C 59/04; A61M 2037/0023; B29C 43/52

(57) ABSTRACT

A mold assembly for forming a microneedle array via variothermal extrusion at a laser-drilled mold.

### TECHNICAL FIELD

The application concerns forming microneedle arrays formed via variothermal extrusion and embossing techniques.

### BACKGROUND

Microneedles are attractive for delivery of certain therapeutics. These needles may be particularly desirable as a mode of therapeutic delivery because of the potential to replace syringe-with-needle type of injections with a pain free alternative. Microneedles can be virtually painless because they do not penetrate deep enough to contact nerves and only penetrate the outermost layer of the skin, unlike traditional syringes and hypodermic needles. Additionally, a less shallow penetration may also reduce the risk of infection or injury. Microneedles may also facilitate delivery of a more precise dosage of a therapeutic which enables the use of lower doses in treatments. Other advantages of microneedles for drug delivery include the simplified logistics (absence of required cold chain), ability for patient self-administration (no need for doctor, nurse, reduction of people transport). Beyond therapeutic delivery, drug delivery, microneedles have also been investigated for diagnostic applications. Bodily fluids coming out through the punctured skin can be analyzed for e.g. glucose or insulin.

Microneedles often require a manufacturing process that allows mass production at lowest cost, and as a consequence, shortest possible cycle time. To have proper transcription of mold texture and shape to the molded part, high flow may be necessary, especially having low viscosity at extremely high shear rates. Furthermore, good release from the production mold is important to reduce cycle time to improve the cost efficiency. The needles formed therefrom should exhibit good strength to prevent breaking of the microneedle during usage. While there are a number of benefits to the use of microneedles and considerations with respect to forming them, certain challenges remain in microneedle production. It would be beneficial to prepare microneedles that exhibit a certain aspect ratio for a sharp tip and blade to puncture the skin.

### SUMMARY

Aspects of the present disclosure concern a method of forming a mold for a microneedle array comprising: forming one or more depressions at a surface of a band mold via laser percussive drilling; disposing the band mold adjacent a chill roll, the chill roll configured to facilitate thermal management of the band mold; depositing a substrate at the band mold; and deforming the substrate into one or more depressions at the band mold.

Other aspects concern a method of forming a microneedle array comprising: directing a pulsed laser along a portion of a band mold to form one or more depressions at a surface of the band mold; disposing the band mold adjacent a chill roll, the chill roll configured to facilitate thermal management of the band mold; depositing a material onto a surface of the band mold, while the band mold is disposed adjacent the chill roll; applying heat to the band mold at or adjacent a point of contact between the material and the band mold, wherein the chill roll operates as a heat sink during the applying of heat; causing the material to move into the depressions of the band mold thereby forming one or more projections at the surface of the material wherein the projections correspond to the depressions of the band mold; and demolding the material from the surface of the band mold.

### BRIEF DESCRIPTION OF THE DRAWINGS

FIG. 1 provides a depiction of exemplary types of embossing processes.

FIG. 2 provides a schematic diagram of a variothermal embossing process according to an aspect of the present disclosure.

FIG. 3 provides a bright field analysis for cylindrical drilling with 100 μm diameter sample.

FIG. 4 provides a bright field analysis for laser percussion drilling sample.

FIG. 5 provides a bright field analysis for cylindrical drilling and point and shoot standing laser.

### DETAILED DESCRIPTION OF ILLUSTRATIVE EMBODIMENTS

The present disclosure can be understood more readily by reference to the following detailed description of the disclosure and the Examples included therein.

Microneedles can be used to deliver a therapeutic or to draw blood without penetrating tissue as deep a traditional needles. Such microneedles can be used individually or as an array of needles. The needles are typically produced via mass production at a low cost. To efficiently function as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while the formed needles still maintain their structural integrity and strength. The mold assembly and methods of forming thereof may provide a microneedle array having the desired varying aspect ratio sufficient to provide a sharp tip among the microneedles and a sharp blade to properly penetrate or cut the skin. The mold assembly for forming a microneedle array may comprise a laminate mold portion and a base mold portion configured to form a microneedle array wherein at least a portion of the microneedles vary in size relative to each other.

According to aspects of the present disclosure, a method of forming a microneedle array may comprise directing a pulsed laser along a portion of a band mold to form one or more depressions at a surface of the band mold; disposing the band mold adjacent a chill roll, the chill roll configured to facilitate thermal management of the band mold; depositing a material onto a surface of the band mold, while the band mold is disposed adjacent the chill roll; applying heat to the band mold at or adjacent a point of contact between the material and the band mold, wherein the chill roll operates as a heat sink during the applying of heat; causing the material to move into the depressions of the band mold thereby forming one or more projections at the surface of the material wherein the projections correspond to the depressions of the band mold; and demolding the material from the surface of the band mold. In further aspects, a method of forming a mold for a microneedle array, the method comprising forming a plurality of conical depressions at a surface of a band mold via laser percussion drilling. The formed mold may be coupled to a variothermal molding apparatus to facilitate formation of a microneedle array.

Forming a microneedle array may comprise a process of forming a plurality of depressions at a surface of a band mold. A band mold may comprise a hollow cylindrical substrate which may be so sized to receive a chill roll or cooling roll within its hollow interior. As an example the band roll may have a diameter of about 144 millimeters (mm) and a surface width of about 350 mm. The band roll may have a thickness of up to about 3 mm to accommodate the depressions corresponding to microneedle structures formed therein. In one example, the band mold may have a thickness of about twice that of the depth of the depressions corresponding to microneedle structures formed therein. Larger values for the thickness of the band mold may prevent sufficient malleable or bending of the band mold to fit the chill roll. In various aspects, the band mold may comprise a metal substrate such as steel, specifically, steel tempered at about 500° C. Other exemplary materials for forming the band roll may comprise copper, and zinc, as well as alloys and semi-alloys. Generally, the band mold may comprise a material that is flexible, is compatible with metal plating, and/or can withstand laser drilling or ablation to form a precise conical depression. The band mold may have a particular surface roughness (Rₘₐₓ) for formation of the microneedle structure therein. The band mold may have a surface roughness of about 3 to about 5 micrometers (μ).

Laser ablation or drilling may be used to form the plurality of depressions at an exterior surface of the band mold. Laser drilling may produce small (e.g., 100 μm) diameter depressions, depressions with high aspect ratios (depth to diameter; ratio greater than about 10:1), and depressions at shallow (10°) angles from the surface. The depressions formed via laser drilling may have a conical geometry corresponding to the geometry of a needle. Laser drilling may comprise focusing a high power laser beam onto the surface of the band mold. At least a portion of the laser beam may be absorbed at the band mold; the amount of absorption may depend upon the material comprising the band mold and condition of the surface of the band mold. As an example, a high intensity (on the order of 10⁷ watts per square centimeter) produced by absorption of high power (e.g., 250 watts) and focusing the beam to 100 μm to 200 μm diameter may result in heating, melting, and vaporization of a surface material.

The laser drilling described herein may comprise laser percussion drilling wherein a stationary laser beam and one or more pulses penetrate a surface of the band mold to form a depression. With percussion drilling, the depression diameter may be determined by the beam diameter and power level. In various aspects, the laser percussion drilling to form conical depressions in the band mold may be performed using a pulsed yttrium aluminum garnet (YAG) laser. The laser may generate a beam of about 100 μm to about 300 μm in diameter and a depth of about 300 μm to about 800 μm at an average energy of about 250 watts (W). As an example, the laser may be stationary, as in point and shoot percussion drilling, or in motion, as in cylindrical laser percussion drilling.

Formation of a microneedle array with the band mold may be achieved by a number of molding processes configured to deposit a material into the conical depressions of the band mold. In various aspects, the band mold may be fitted to or disposed at a roll of an embossing process. Embossing may be used to impart a texture or pattern into a number of products including textiles, paper, synthetic materials, metals, wood, and polymeric materials. In an embossing process, a substrate is caused to conform under pressure to the depths and contours of a pattern engraved or otherwise formed on an embossing roll. Embossing may be accomplished by passing a substrate through one or more patterned embossing rolls set to apply a certain pressure and penetration depth to the substrate. As the substrate passes the embossing rolls, the pattern on the one or more rolls is imparted onto the substrate.

The patterns on the embossing rolls may be mated or non-mated. In a pair of mated embossing rolls, the pattern on one of the rolls may identically, or similarly, compliment, or “mate,” with the pattern on a second or other of the mated rolls. The pattern on a non-mated embossing roll does not match identically with the pattern on the other roll. Depending on the desired results, either type of embossing roll can be used.

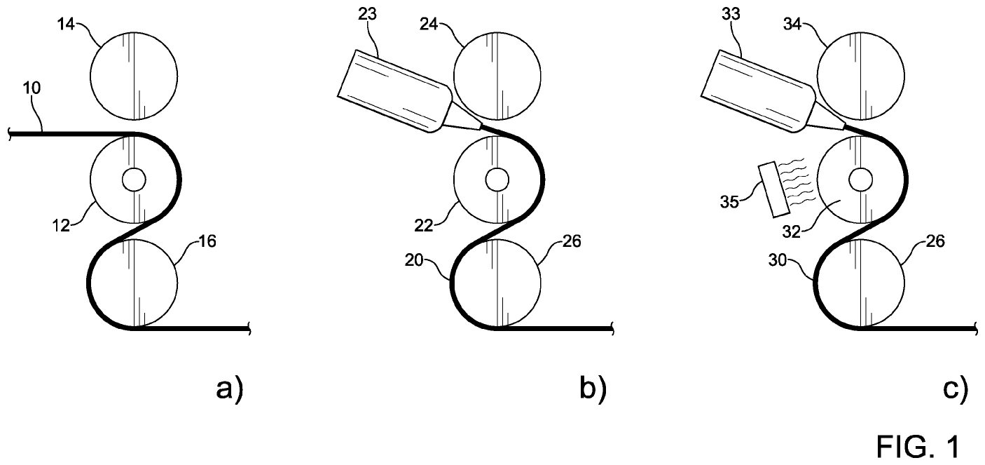

Various types of embossing processes may be useful in the formation of a microneedle array according to the methods described herein. These extrusion types may include, for example, roller embossing, extrusion embossing, and a variant on extrusion embossing referred to as variothermal extrusion embossing as shown in FIG. 1 as (a), (b), and (c), respectively. In a roller embossing process (a), a substrate (in the form of a sheet) 10 may be contacted with a rotating chill roll 12 having protrusions or depressions on a surface thereof. The chill roll 12 may be so named for its function of allowing the embossed substrate to cool and set into its embossed shape and may also be referred to as an embossing roll. A counterpressure roll 14 may rotate to apply a force to depress the substrate 10 into depressions or protrusions on the surface of the chill roll 12 thereby delivering a reciprocal pattern onto the substrate 10. As described above, the chill roll 12 and the counterpressure roll 14 may be “mated” or “non-mated.” A receiving roll 16 receives a now embossed substrate 10 as it peels off of the chill roll 12. The embossed substrate 10 from a roller embossing process may be thermally or ultraviolet (UV) cured and the process enables a continuous production of polymeric films. Replication time (or formation of an embossed substrate) may be limited as a function of the rotation speed and diameter of the embossing roll. Pressure is generally not applied as the embossed substrate travels to the receiving roll in order to compensate against shrinkage. Demolding from the chill roll in roller embossing may occur with a peeling movement. Roller embossing may be advantageous for its production speed which can reach 60 meters per minute by a 2 meter width. The formation of deep structures as well as high-aspect ratios and replication quality however may present a challenge.

In extrusion embossing (b), a substrate 20 may be applied to a surface of the chill roll 22 via a direct feed from an extrusion die apparatus 23. Thus, the substrate 20 need only be used in material pellet form rather than forming a sheet prior to embossing. A rotating counterpressure roll 24 may be used to apply a force at the extruded substrate 20 and a receiving roll 26 receives the substrate as it peels off from the chill roll 22. For the variothermal variant of extrusion embossing (c), a substrate 30 may be applied to a surface of a chill roll 32 via a direct feed from an extrusion die apparatus 33. An external heating source 35 may be applied at an initial point of contact between the substrate 30 and the chill roll to heat the substrate 30 thereby facilitating formation of a reciprocal pattern that corresponds to depressions or protrusions at the heated surface of the chill roll 32. At least a portion of the chill roll 32 may be a cooling section. This cooling section may be disposed at a portion of the chill roll 32 which opposes at least a portion of the area of the chill roll 32 that has been heated by the external heating source. A rotating counterpressure 34 roll may be used to apply a force at the extruded, heated substrate 30 while a receiving roll receives the now embossed substrate 30 as it cools and peels away from the chill roll 32.

According to various aspects of the present disclosure, variothermal embossing combined with the use of a laser-drilled band mold described herein may provide improved production of a microneedle array. Thermal management at a polymer substrate forming the microneedle array may provide a faster production rate because of the increased viscosity of the polymer substrate material while the laser drilled band mold at a chill roll may improve the quality of replication during embossing. The band mold having conical depressions formed at a surface thereof may be disposed adjacent an embossing roll to form a chill roll in an variothermal embossing process. That is, the band mold may be fitted about an embossing roll and configured to receive a substrate. The chill roll may be configured to facilitate thermal management of the band mold. The method of the present disclosure may combine variothermal embossing with a single-step extrusion roller embossing process to provide a microneedle array. The chill roll (including the laser-percussion drilled band mold) may be used as a mold for the microneedle array; the conical depressions of the band mold exhibiting an inverse geometry suitable for microneedles. Variothermal heating may be used to obtain a better heating and cooling distribution on the chill roll thereby facilitating better microneedle replication. Specifically, the external heating source may generate a temperature profile along a circumference of the chill roll which is cooled.

As described herein, a method of forming a microneedle array may comprise directing a pulsed laser along a portion of a band mold to form one or more conical depressions at a surface of the band mold. The band mold may be disposed adjacent, about, around, or on a chill roll in a variothermal extrusion process to form a microneedle array. Forming the microneedle array may comprise forming a mold by directing a pulsed laser along a portion of a band mold to form one or more depressions at a surface of the band mold. The band mold may be disposed adjacent a chill roll wherein the chill roll is configured to cool at least a portion of the band mold. A material or substrate may be deposited on to the band mold and heat may be applied to the band mold at a point of contact between the substrate and the band mold. The substrate may then be caused to move into the depressions of the band mold, thereby forming one or more projections at a surface of the substrate wherein the projections correspond to the depressions of the band mold. The substrate may be demolded from the surface of the band mold to form a microneedle array.

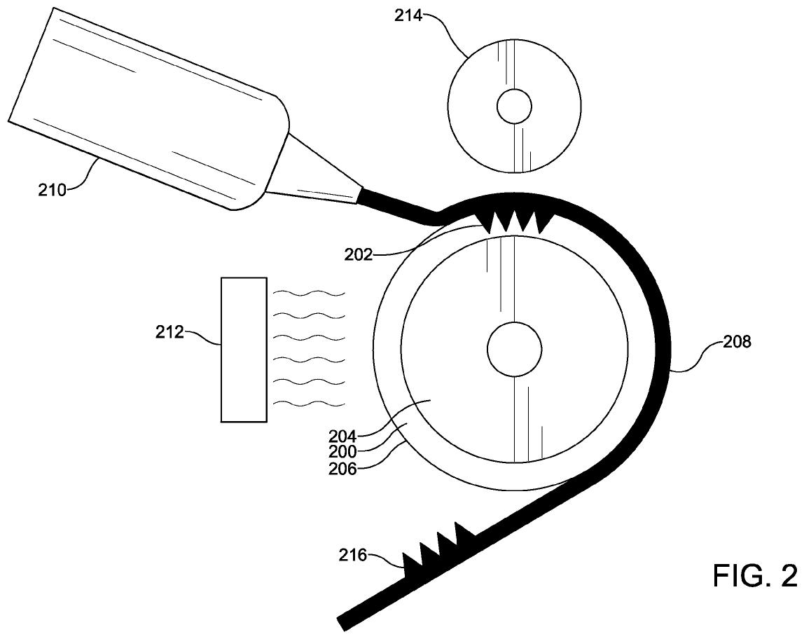

As shown in FIG. 2, to form a microneedle array, a band mold 200 (comprising a plurality of conical depressions 202 formed by laser-drilling at a surface of the band mold 200) may be disposed adjacent an embossing roll 204 to form a chill roll 206. A substrate 208 may be deposited via an extrusion die apparatus 210 at a surface of the band mold 200 comprising the conical depressions 202. While the band mold 200 is disposed adjacent the embossing roll 204, heat may be applied via an external heat source 212 causing the substrate 208 to deform and move into the conical depressions 202 of the band mold 200. Heat may be applied to the band mold 200 at, or adjacent, a point of contact between the substrate 208 and the band mold 200. The chill roll 206 may thus operate as a heat sink by absorbing heat during the application of heat. A counterpressure roll 214 disposed adjacent the band mold 200 may rotate in a direction opposing the band mold 200 thereby advancing the substrate 208 between the band mold 200 and the counterpressure roll 214 and continuing to displacing at least a portion of the substrate 208 into the conical depressions 202. Displacement of the substrate 208 may form one or more projections 216 at the substrate 208 such that the one or more projections 216 correspond to the conical depressions 202 at the band mold 200. The substrate 208 may demold or haul off from the chill roll 206 to provide a microneedle array.

In some aspects, the conical depressions formed in the band mold via laser ablation may be oriented in a specific repeating pattern. In further examples however, the conical depressions may be randomly distributed at the band mold. The orientation of conical depressions in the band mold may thus correspond to a pattern in a resulting microneedle array or may provide a microneedle array in a random configuration.

Heating of at least a portion of the band mold may comprise heating at least a portion of the band mold to a temperature above the melting point of the substrate. While at least once portion of the band mold is heated to cause deformation of the substrate into the conical depressions of the band mold, at least a second portion of the band mold may be maintained at a temperature less than the melting point of the substrate. The chill roll may be configured to be cooled in order to maintain a temperature less than the melting temperature of the substrate. In certain examples, demolding the substrate from the band mold may comprise cooling at least a portion of the band mold via cooling of the chill roll.

In various aspects, the substrate may comprise a polymer material. The substrate for forming a microneedle array in the disclosed variothermal embossing process may comprise a polymer or a mixture of polymers. Generally, the polymer mixture may be supplied in a liquid or flowable state, via for example, an extrusion die apparatus, to the band mold. The solid product comprising the microneedle array may then separate from the band mold. Exemplary polymer materials may comprise engineering thermoplastics such as polycarbonates, polyetherimides, polyphenylene ether, and polybutylene terephthalate, as well as blends of polycarbonate with acrylic butadiene styrene plastics.

The polymer material for forming the microneedle array may further comprise one or more additives intended to impart certain characteristics to a microneedle array formed by the mold assembly described herein. The polymer material may include one or more of an impact modifier, flow modifier, antioxidant, heat stabilizer, light stabilizer, ultraviolet (UV) light stabilizer, UV absorbing additive, plasticizer, lubricant, antistatic agent, anti-fog agent, antimicrobial agent, colorant (e.g., a dye or pigment), surface effect additive, radiation stabilizer, anti-drip agent (e.g., a polytetrafluoroethylene(PTFE)-encapsulated styrene-acrylonitrile copolymer (TSAN)), or a combination comprising one or more of the foregoing. For example, a combination of a heat stabilizer and ultraviolet light stabilizer can be used. In general, the additives are used in the amounts generally known to be effective. For example, the total amount of the additive composition can be 0.001 to 10.0 weight percent (wt %), or 0.01 to 5 wt %, each based on the total weight of all ingredients in the composition.

The polymer material may include various additives ordinarily incorporated into polymer compositions, with the proviso that the additive(s) are selected so as to not significantly adversely affect the desired properties of the thermoplastic composition (good compatibility for example). Such additives can be mixed at a suitable time during the mixing of the components for forming the composition.

In addition, the polymer material may exhibit excellent release, as measured by ejection force (in newtons, N) and coefficient of friction. The polymer material also preferably show (i) high flow at high shear conditions to allow good transcription of mold texture and excellent filling of the finest mold features, (ii) good strength and impact (as indicated by ductile Izod Notched Impact at room temperature and modulus), and (iii) high release to have efficient de-molding and reduced cooling and cycle time during molding. The microneedles formed herein may have sufficient mechanical strength to remain intact (i) while being inserted into the biological barrier, (ii) while remaining in place for up to a number of days, and (iii) while being removed.

The microneedle array of the present disclosure may fulfill regulatory critical to quality (CTQ) requirements. There should be minimal or no chemical reaction among the active ingredient of the therapeutic, the carrier/coating, and the material forming the microneedle array during production, sterilization, storage, and/or during the use of the microneedle array. Such interactions may destroy or alter the active ingredient, affect needle properties, or both.

### Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “weight %,” and “wt %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {M}_{i}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si—O—Si linkage.

The term “flowable” means capable of flowing or being flowed. Typically a polymer is heated such that it is in a melted state to become flowable. ° C. is degrees Celsius. μm is micrometer. cS is centistroke. kG is kilogram.

### Aspects

The present disclosure comprises at least the following aspects.

Aspect 1A. A method of forming a mold for a microneedle array comprising: forming one or more depressions at a surface of a band mold via laser percussive drilling; disposing the band mold adjacent a chill roll, the chill roll configured to facilitate thermal management of the band mold; depositing a substrate at the band mold; and deforming the substrate into one or more depressions at the band mold.

Aspect 1B. A method of forming a mold for a microneedle array consisting essentially: forming one or more depressions at a surface of a band mold via laser percussive drilling; disposing the band mold adjacent a chill roll, the chill roll configured to facilitate thermal management of the band mold; depositing a substrate at the band mold; and deforming the substrate into one or more depressions at the band mold.

Aspect 1C. A method of forming a mold for a microneedle array consisting: forming one or more depressions at a surface of a band mold via laser percussive drilling; disposing the band mold adjacent a chill roll, the chill roll configured to facilitate thermal management of the band mold; depositing a substrate at the band mold; and deforming the substrate into one or more depressions at the band mold.

Aspect 2. A method of forming a microneedle array, the method comprising: directing a pulsed laser along a portion of a band mold to form one or more depressions at a surface of the band mold; disposing the band mold adjacent a chill roll, the chill roll configured to facilitate thermal management of the band mold; depositing a material onto a surface of the band mold, while the band mold is disposed adjacent the chill roll; applying heat to the band mold at or adjacent a point of contact between the material and the band mold, wherein the chill roll operates as a heat sink during the applying of heat; causing the material to move into the depressions of the band mold thereby forming one or more projections at the surface of the material wherein the projections correspond to the depressions of the band mold; and demolding the material from the surface of the band mold.

Aspect 2. A method of forming a microneedle array, the method consisting essentially of: directing a pulsed laser along a portion of a band mold to form one or more depressions at a surface of the band mold; disposing the band mold adjacent a chill roll, the chill roll configured to facilitate thermal management of the band mold; depositing a material onto a surface of the band mold, while the band mold is disposed adjacent the chill roll; applying heat to the band mold at or adjacent a point of contact between the material and the band mold, wherein the chill roll operates as a heat sink during the applying of heat; causing the material to move into the depressions of the band mold thereby forming one or more projections at the surface of the material wherein the projections correspond to the depressions of the band mold; and demolding the material from the surface of the band mold.

Aspect 2. A method of forming a microneedle array, the method comprising: directing a pulsed laser along a portion of a band mold to form one or more depressions at a surface of the band mold; disposing the band mold adjacent a chill roll, the chill roll configured to facilitate thermal management of the band mold; depositing a material onto a surface of the band mold, while the band mold is disposed adjacent the chill roll; applying heat to the band mold at or adjacent a point of contact between the material and the band mold, wherein the chill roll operates as a heat sink during the applying of heat; causing the material to move into the depressions of the band mold thereby forming one or more projections at the surface of the material wherein the projections correspond to the depressions of the band mold; and demolding the material from the surface of the band mold.

Aspect 3. The method of any of aspects 1A-2C, wherein at least a portion of the one or more of the depressions are conical.

Aspect 4. The method of any of aspects 1A-3, wherein at least a portion of the one or more depressions have a conical shape.

Aspect 5. The method of any of aspects 1A-4, wherein at least a portion of the one or more of the depressions have a half pyramidal geometry.

Aspect 6. The method of any one of aspects 1A-5, wherein the one or more depressions are formed via point and shoot with standing laser percussion drilling.

Aspect 7. The method of any one of aspects 1A-5, wherein the one or more depressions are formed via cylindrical motion laser percussion drilling.

Aspect 8. The method of any one of aspects 1A-5, wherein the one or more depressions are formed via a combination of point and shoot laser percussion drilling and cylindrical laser percussion drilling.

Aspect 9. The method of any one of aspects 3-8, wherein the depositing the material comprises extruding the material.

Aspect 10. The method of any one of aspects 3-9, wherein the one or more projections formed at the surface of the material are formed in a pattern.

Aspect 11. The method of aspect 10, wherein the one or more projections formed at the surface of the material are formed in a pattern corresponding to a microneedle array.

Aspect 12. The method of any one of aspects 3-11, wherein the one or more projections at the surface of the material are formed in a random configuration.

Aspect 13. The method of any one of aspect 3-12, wherein the material comprises a thermoplastic.

Aspect 14. The method of any one of aspects 3-13, wherein the band mold comprises steel.

Aspect 15. The method of any one of aspects 3-14, wherein the band mold comprises an alloy.

Aspect 16. The method of any one of aspects 3-15, wherein the causing the material to move into the one or more depressions comprises engaging the chill roll having the band mold disposed there at and a counter pressure roll disposed adjacent band mold to rotate in opposing directions thereby advancing the material between the band mold and the counter pressure roll and displacing at least a portion of the material into at least a portion of the one or more depressions.

Aspect 17. The method of any one of aspects 3-16, wherein the heating of the band mold comprises heating at least a portion of the band mold to a temperature above a melting temperature of the material.

Aspect 18. The method of any one of aspects 3-17, wherein a temperature of at least a second portion of the band mold is maintained, via the chill roll, to be less than a melting temperature of the material.

Aspect 19. The method of any one of aspects 3-18, wherein demolding the material from the surface of the band mold roll comprises cooling at least a portion of the band mold via the chill roll.

Aspect 20A. A microneedle array formed by a method comprising: directing a pulsed laser along a portion of a band mold to form one or more depressions at a surface of the band mold; disposing the band mold to a chill roll wherein the chill roll is configured to cool at least a portion of the band mold; depositing a material onto the band mold; applying heat to the band mold at a point of contact between the material and the band mold; causing the material to move into the depressions thereby forming one or more projections at a surface of the material wherein the projections correspond to the depressions of the band mold; and demolding the material from the surface of the band mold.

Aspect 20B. A microneedle array formed by a method consisting essentially of: directing a pulsed laser along a portion of a band mold to form one or more depressions at a surface of the band mold; disposing the band mold to a chill roll wherein the chill roll is configured to cool at least a portion of the band mold; depositing a material onto the band mold; applying heat to the band mold at a point of contact between the material and the band mold; causing the material to move into the depressions thereby forming one or more projections at a surface of the material wherein the projections correspond to the depressions of the band mold; and demolding the material from the surface of the band mold.

Aspect 20C. A microneedle array formed by a method consisting of: directing a pulsed laser along a portion of a band mold to form one or more depressions at a surface of the band mold; disposing the band mold to a chill roll wherein the chill roll is configured to cool at least a portion of the band mold; depositing a material onto the band mold; applying heat to the band mold at a point of contact between the material and the band mold; causing the material to move into the depressions thereby forming one or more projections at a surface of the material wherein the projections correspond to the depressions of the band mold; and demolding the material from the surface of the band mold.

Aspect 21. The microneedle array of aspect 20, wherein the depressions are conical.

Aspect 22. The microneedle array of any one of aspects 20-21, wherein the depressions of the band mold correspond

Aspect 23. The microneedle array of any one of aspects 20-22, wherein the material comprises a thermoplastic.

Aspect 24. The microneedle array of any one of aspects 20-23, wherein the microneedle array exhibits varying aspect ratios.

Aspect 25. The microneedle array of any one of aspects 20-24, wherein the band mold comprises steel.

### EXAMPLES

The disclosure may be illustrated by the following non-limiting examples.

The patterning technology for forming conical depressions in a stainless steel plate was evaluated by testing on a flat metal surface. Laser percussion drilling was performed on a flat plate (70 millimeter (mm)×70 mm×20 mm mold carbon steel 1.1730) in a series of three columns along the surface of the steel plate according to the processing conditions of Table 1.

TABLE 1
Processing conditions for laser percussion drilling

| | |
|---|---|
| Colum 1 | Cylindrical drilling with 100 μm in diameter |
| Colum 2 | “Point-and-shoot” (standing laser). 2.1 + 2.2 |
| | variation of Laser-on-time, 2.3-2.8 Variation of focus |
| Colum 3 | Combination cylindrical drilling und point-and-shoot |

The testing setup included standard drilling with a 100 μm diameter (Column 1), a point-and-shoot drilling with standing laser (Column 2), and a combination of cylindrical drilling and point-and-shoot was performed (Column 3). Columns 2 and 3 represent the laser percussion drilling described in the present disclosure. Point and shoot as used for column 2 refers to a static or stationary laser. Column 3 also applies point and shoot, but the laser also moves in a circular motion. For Column 2, a variation of laser-on-time and variation of focus was performed. High high-aspect-ratio depressions and depressions with a depth-to-diameter ratio much greater than 10:1 were formed. Before laser percussion drilling, the steel plate appeared to have a smooth uniform appearance. After the drilling, the surface of the steel plate had a number of depressions or conical indentations.

Analysis of the conical depressions formed by the laser drilling was analyzed using light microscopy. A bright field analysis of each array of depressions was performed with a depth profile to determine if the depressions have an appropriate geometry for use in a microneedle array. Table 4 provides the instrumentation and conditions used.

TABLE 2
Instrumentation and conditions for analysis of depressions

| | |
|---|---|
| Microscopy Instruments | LM: Keyence VX5500 |
| Experimental Conditions | LM: bright field and depth profile. |
| Keywords and Comments | Holes |

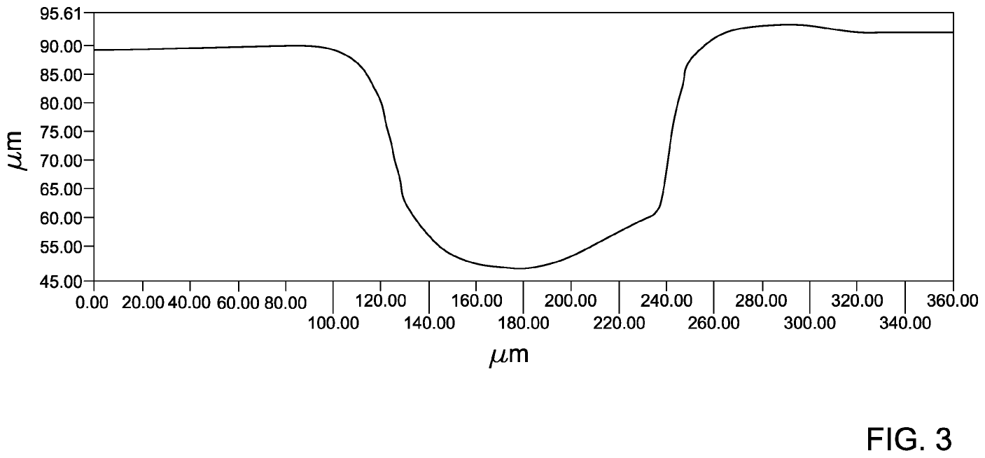

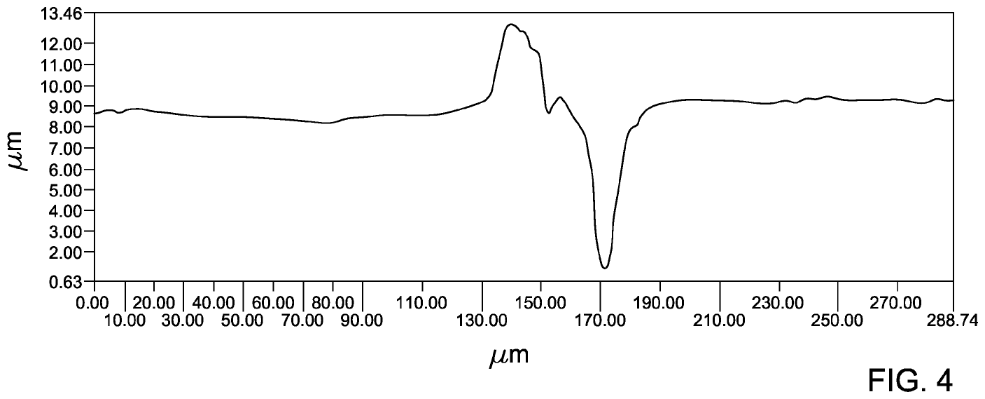

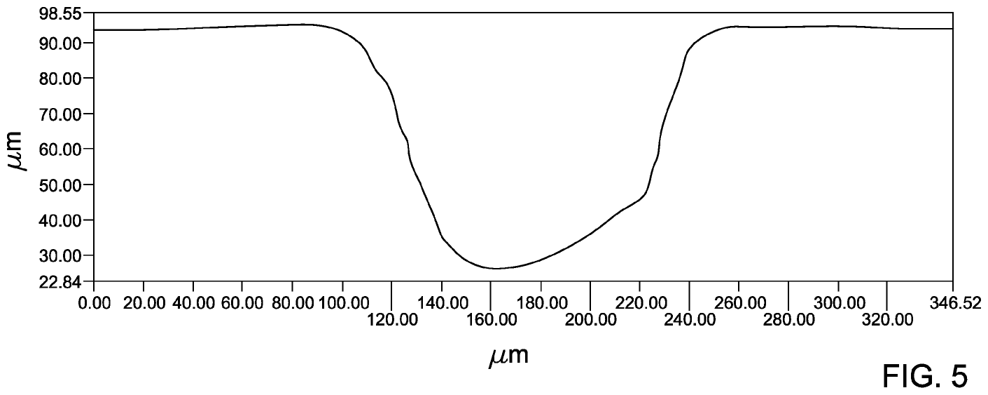

FIGS. 3-5 provide the bright field analysis for a depression of Column 1, Column 2, and Column 3, respectively. The analyses of the depressions corresponding to Column 1 (i.e., cylindrical drilling with 100 μm diameter) and Column 3 (i.e., combination cylindrical drilling and point and shoot) revealed the depressions have a round shape. The depth analysis further indicated that drilling can provide a more cone shaped hole, which is desired for the microneedle shape. The depressions of C 2 (i.e., “point-and-shoot” (standing laser)) were not as circular. Some of the depressions of column 2 also exhibited a sharp conical shape resembling a microneedle structure. Diameter measurements are summarized in Table 3.

TABLE 3
Diameter measurements for conical depressions of Column 1, 2, or 3

| | Diameter | | Diameter | | | Diameter |
|---|---|---|---|---|---|---|
| Column | (μm) | Column | (μm) | Shape | Column | (μm) |
| 1.1 | 153 | 2.1 | 53 | not | 3.1 | 153 |
| | | | | round | | |
| 1.2 | 157 | 2.2 | 58 | not | 3.2 | 156 |
| | | | | round | | |
| 1.3 | 156 | 2.3 | 137 | not | 3.3 | 158 |
| | | | | round | | |
| 1.4 | 160 | 2.4 | 125 | not | 3.4 | 165 |
| | | | | round | | |
| — | — | 2.5 | 92 | not | — | — |
| | | | | round | | |
| — | — | 2.6 | 87 | not | — | — |
| | | | | round | | |
| — | — | 2.7 | 120 | not | — | — |
| | | | | round | | |
| — | — | 2.8 | 130 | not | — | — |
| | | | | round | | |

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of forming a mold for a microneedle array comprising:
forming one or more depressions at a surface of a band mold via laser percussive drilling;
disposing the band mold adjacent a chill roll, the chill roll configured to facilitate thermal management of the band mold;
depositing a substrate at the band mold; and
deforming the substrate into one or more depressions at the band mold.

2. A method of forming a microneedle array comprising:
directing a pulsed laser along a portion of a band mold to form one or more depressions at a surface of the band mold;
disposing the band mold adjacent a chill roll, the chill roll configured to facilitate thermal management of the band mold;
depositing a material onto a surface of the band mold, while the band mold is disposed adjacent the chill roll;
applying heat to the band mold at or adjacent a point of contact between the material and the band mold, wherein the chill roll operates as a heat sink during the applying of heat;
causing the material to move into the depressions of the band mold thereby forming one or more projections at a surface of the material wherein the projections correspond to the depressions of the band mold; and
demolding the material from the surface of the band mold.

3. The method of claim 2, wherein at least a portion of the one or more of depressions are conical.

4. The method of claim 3, wherein at least a portion of the one or more of depressions have a half pyramidal geometry.

5. The method of claim 3, wherein the depositing the material comprises extruding the material.

6. The method of claim 3, wherein the one or more projections formed at the surface of the material are formed in a pattern.

7. The method of claim 6, wherein the one or more projections formed at the surface of the material are formed in a pattern corresponding to a microneedle array.

8. The method of claim 3, wherein the one or more projections at the surface of the material are formed in a random configuration.

9. The method of claim 3, wherein the material comprises a thermoplastic.

10. The method of claim 3, wherein the causing the material to move into the depressions comprises engaging the chill roll having the band mold disposed there at and a counter pressure roll disposed adjacent band mold to rotate in opposing directions thereby advancing the material between the band mold and the counter pressure roll and displacing at least a portion of the material into at least a portion of the one or more depressions.

11. The method of claim 3, wherein the heating of the band mold comprises heating at least a portion of the band mold above a melting temperature of the material.

12. The method of claim 3, wherein a temperature of at least a second portion of the band mold is maintained, via the chill roll, to be less than a melting temperature of the material.

13. The method of claim 3, wherein demolding the material from the surface of the band roll comprises cooling at least a portion of the band mold via the chill roll.

14. A microneedle array formed by a method comprising:
directing a pulsed laser along a portion of a band mold to form one or more depressions at a surface of the band mold;
disposing the band mold to a chill roll wherein the chill roll is configured to cool at least a portion of the band mold;
depositing a material onto the band mold;
applying heat to the band mold at a point of contact between the material and the band mold;
causing the material to move into the depressions thereby forming one or more projections at a surface of the material wherein the projections correspond to the depressions of the band mold; and demolding the material from the surface of the band mold.

15. The microneedle array of claim 14, wherein the one or more depressions are conical.

16. The microneedle array of claim 14, wherein the one or more depressions of the band mold correspond to a configuration for a microneedle array.

17. The microneedle array of claim 14, wherein the material comprises a thermoplastic.

18. The microneedle array of claim 14, wherein the microneedle array exhibits varying aspect ratios.
